(19) United States
(12) Patent Application Publication
(10) Pub. No.: US 2005/0101033 A1
(43) Pub. Date: May 12, 2005

(54) Measurement system, method and program for measuring refinement rate of competitive system

(75) Inventor: Hiroyuki Iida, Hamamatsu City (JP)

Correspondence Address: OLIFF & BERRIDGE, PLC, P.O. BOX 19928, ALEXANDRIA, VA, 22320, US

(73) Assignee: National University Corporation Shizuoka University, Shizuoka City (JP)

(21) Appl. No.: 10/956,122

(22) Filed: Oct. 4, 2004

(30) Foreign Application Priority Data
Oct. 9, 2003 (JP) 2003-350,543

(51) Int. Cl. A63F 9/24; A63F 13/00; G06F 17/00; G06F 19/00; G01N 33/48; G01N 33/50

(52) U.S. Cl. 436/518; 702/19; 463/9

(57) ABSTRACT

There is provided a measurement system for measuring a refinement rate of a competitive system, the system includes: input part for inputting competition data of the competitive system, said competition data, obtained by users between a beginning and end of the competition, including transition in information amount necessary to know a conclusion of the competition; first calculating part for obtaining an average completion time length D and an average degree of freedom B of the user and from said inputted competition data; and second calculating part for calculating a refinement rate S based on said obtained B and D.

### BACKGROUND OF THE INVENTION

1. Field of the Invention

The present invention relates to a measurement system, method and program for measuring refinement rate of competitive system.

2. Related Art Statements

Western chess, Shogi (Japanese chess), Xiangqi (Chinese chess), Go (game of go) and the like have been popular and they have been played by people all over the world from long ago to present day. However, there is no index or rating for representing which the most complicated game among them is, such as an index indicating complexity, entertaining, and degree of refinement of a game. In the more general sense, there does not exist any technique for rationalistically and objectively measuring or evaluating both a refinement degree of an entertainment system, which can be regarded as a competitive sport or competitive system, and a skill level of the player group. If there is a technique to evaluate or rate such a refinement degree of a competitive system, it is possible to simulate how a refinement rate (i.e., entertaining, amusingness or a fascinating aspect) changes by altering or modifying rules of a competitive system.

In a field of competitive systems such as games there is a conventional technique, which is called a “rating system” and is well known, for evaluating an abilities or skills of a person based on statistical information. In the field of chess Professor Arpad Elo developed Elo Rating System (refer to a document: Arpad Elo, “The Rating Chessplayers, Past and Present” published by Arco Publishers, 1978) and then this rating system is very well known in the art. This conventional rating system aims for evaluation of individual's ability or personal game playing skills, the system cannot evaluate or rate a refinement degree or entertaining of a competitive system itself or a skill level of a certain user group.

In this connection, the present inventor has filed a Japanese patent application (JPA 2003-187016) for a method of evaluating abilities of a player. This method is an evaluating skills by providing a player with some question to prompt him/her to make answers, but this method cannot evaluate or rate a refinement degree or amusingness of a competitive system itself or a skill level of a certain user group by processing past competition data (i.e., game records) statistically.

### SUMMARY OF THE INVENTION

It is an object of the present invention to provide a measurement system, method and program for measuring a refinement rate of a competitive system based on past competitive information.

In order to solve the above-mentioned problems, there is provided a measurement system for measuring a refinement rate of a competitive system, said system comprises:
an input means for inputting competition data of the competitive system, said competition data, obtained by users between a beginning and end of the competition, including transition in information amount (i.e., uncertainty) necessary to know a outcome (i.e., winning or losing) of the competition;
first calculating means for obtaining an average completion time length D and an average degree of freedom B of the user and from said inputted competition data; and
second calculating means for calculating a refinement rate S based on said obtained B and D.

According to the present invention, refinement rates for various competitive systems can rationalistically and objectively be provided and the refinement rates for various competitive systems can be evaluated and compared as indexes for representing a refinement level, entertaining, amusingness, fascinating or the like.

In an embodiment of the measurement system according to the present invention, said second calculation means calculates the refinement rate S using following equations:
S=B/D² or S=B^{0.5}/D.

In another embodiment of the measurement system according to the present invention,
said input means inputs the competition data by each user group (player group),
and said second calculating means calculates said refinement rate S as a skill level J by each user group.

Assuming that refinement or sophistication of respective competitive systems are identical with each other, the sophistication rates S can be treated as skill levels J of respective user groups who participate in the competition. Furthermore, skill level differences among different groups in the same competition, such as a masters player group, an amateur group, and a student group, can be expressed as numerical values and the values can be compared.

In yet another embodiment of the measurement system according to the present invention,
said competitive system is a board game; said average degree of freedom B is an average number of possible/legal moves between the beginning and the end of the game; and
said average completion time length D is an average number of actual moves played in the game (i.e., between the beginning and the end of the game).

By way of easy explanation the aspect of the present invention has been described as the systems (i.e., devices), however it is understood that the present invention may be realized as methods corresponding to the systems, programs embodying the methods as well as a storage media storing the programs therein.

For example, according to another aspect of the present invention, there is provided a measurement method for measuring a refinement rate of a competitive system, the method comprises the steps of:
inputting competition data of the competitive system, said competition data, obtained by users between a beginning and end of the competition, including transition in information amount necessary to know a conclusion (i.e., winning or losing) of the competition;
obtaining an average completion time length D and an average degree of freedom B of the user and from said inputted competition data using a calculating means; and
calculating a refinement rate S based on said obtained B and D using a calculating means.

In an embodiment of the measurement method according to the present invention, said calculating step calculates the refinement rate S using following equations:
S=B/D² or S=B^{0.5}/D.

In another embodiment of the measurement method according to the present invention,
said inputting step inputs the competition data by each user group,
and said calculating step calculates said refinement rate S as a skill level J by each user group.

In yet another embodiment of the measurement method according to the present invention,
said competitive system is a board game;
said average degree of freedom B is an average number of possible moves between the beginning and the end of the game; and
said average completion time length D is an average number of actual moves played in the game.

In addition, according to still another aspect of the present invention, there is provided a measurement program for executing a measurement method for measuring a refinement rate of a competitive system, the program comprises the steps of:
inputting competition data of the competitive system, said competition data, obtained by users between a beginning and end of the competition, including transition in information amount necessary to know a conclusion (i.e., winning or losing) of the competition;
obtaining an average completion time length D and an average degree of freedom B of the user and from said inputted competition data; and
calculating a refinement rate S based on said obtained B and D.

In an embodiment of the measurement program according to the present invention, said calculating step calculates the refinement rate S using following equations:
S=B/D² or S=B^{0.5}/D.

In another embodiment of the measurement method according to the present invention,
said inputting step inputs the competition data by each user group,
and said calculating step calculates said refinement rate S as a skill level J by each user group.

In another embodiment of the measurement method according to the present invention,
said competitive system is a board game;
said average degree of freedom B is an average number of possible moves between the beginning and the end of the game; and
said average completion time length D is an average number of actual moves played in the game.

### BRIEF DESCRIPTION OF THE DRAWINGS

FIG. 1 is a block diagram illustrating a basic configuration of an embodiment of the measurement system for measuring, rating or evaluating a refinement rate of a competitive system;

FIG. 2 is a flow diagram depicting exemplary processing performed by the measurement system according to the present invention; and

FIG. 3 is a graph showing exemplary logistic model representing information transition of a game.

### DETAILED DESCRIPTION OF THE PREFERRED EMBODIMENTS

Several preferred embodiments of the present invention will be described with reference to the accompanying drawings.

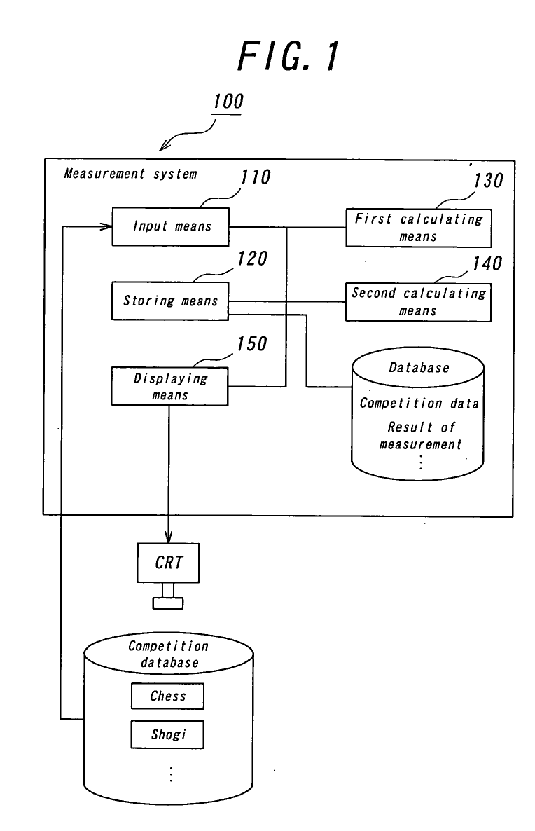

FIG. 1 is a block diagram illustrating a basic configuration of an embodiment of the measurement system for measuring, rating or evaluating a refinement rate of a competitive system. As shown in FIG. 1, a measurement system 100 comprises:
an input means 100 for inputting competition data of the competitive system, said competition data, obtained by users between a beginning and end of the competition, including transition in information amount necessary to know a conclusion of the competition;
a storing means (i.e., storage) 120 for storing the inputted competition data therein;
a first calculating (processing) means 130 for obtaining an average completion time length D and an average degree of freedom B of the user and from said inputted competition data;
a second calculating (processing) means 140 for calculating a refinement rate S based on said obtained B and D; and
a displaying means (i.e., a display) 150 for displaying the calculated refinement rate S, the obtained data, and the like thereon.

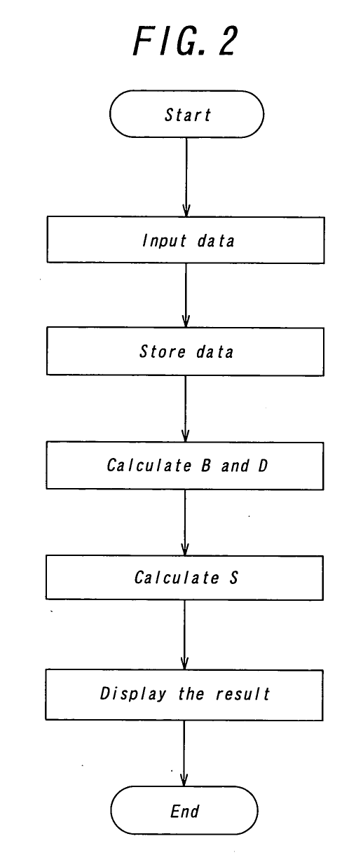

FIG. 2 shows a process carried out by the above described measurement system as a flow chart. In the drawing, each of the illustrated process boxes (steps) can be relocated anywhere if not logically contradictory.

In the present invention, a competitive system is perceived as a framework consisting of two elements: degree of freedom of a user (in the field of the game which is often called a player); and a competition time length (i.e., a time length between a beginning and end of the competition). In the present invention, a logistic model, which will be explained in detail later, is utilized for solving a system (in the game field, a resolution is equivalent to completely knowing result of win-or-lose) in a deterministic way. The model is based on certainty, which is quantified as the degree of freedom. In an approach of the present invention, it is considered that the later stage of the competition a competitive system is resolved in deterministic approach by users, the higher degree of refinement the system has. In other words, in the present invention it is assumed that a degree of a competition system corresponds to a value (which is equivalent to an accelerated velocity), in which an equation of a logistic model is differentiated twice with respect to time. Thus in the present invention this value is used as a reference index to the refinement rate.

Therefore, the sophistication rate S proposed by the present invention may be used for measuring sophistication degrees of various entertainment systems. In the present invention, if there are some kinds of user group data (including a degree of freedom and a time between start and goal), each skill level of each user group can be compared with others. Namely, according to the present invention, in a competition system as an entertainment system (including competitive sports, and board games and all the like), a sophistication rate or a skill level of users in that system can be evaluated or measured in relative or absolute terms. In addition, the present invention is applicable to various fields, for example, when some indexes derived by the present system are used as references, it becomes possible that new entertainment system can be designed or existing competitive system may be improved using the indexes provided this invention.

Hereafter, based on the past competition or game data of the board games, such as shogi and chess, which are the thinking games which can be qualified as traditional and classical entertainment systems, it will be explained that how the measurement system according to the present invention actually measures and estimates the degree of refinement or sophistication of each game.

Firstly, although a person intuitively feels that a game is more refined, sophisticated or profound, it can be considered that the following reasons for that feeling are essential.

1. The quantity of the flexibility or freedom given to respective players, i.e., the indefinite nature with which a game provides a player, is a considerable scale. That is, when the degree of flexibility or freedom is too large it is useless, and it is useless if the flexibility or freedom is too small. A size with a sufficient degree is important.

2. The end (victory and defeat) of a game is not known to the last. Amusingness will be lost if complexion of a game is bound so that it cannot turn back.

A Logistic Model of a Game

It is assumed that the vertical axis denotes an amount of information x(t), which is needed for deterministically knowing the conclusion (i.e., winning or losing) of a game and the horizontal axis denotes time t. It is also assumed that B denotes an average number of possible/legal moves between the beginning and the end of the game and D denotes an average number of actual moves played in the game. In this circumstance, a logistic model regarding amount of information acquired by players as time advances that is the game progresses is
given by the following equation:
x(t)=B(t/D)ⁿ equation (1)
where nε=R (real number)

When the equation is applied to board games this equation means that a certain moves is selected from a set of possible moves by a player and thus a conclusion of the game becomes gradually clear as time progresses. When a game is played by two players in which there is a large skill difference therebetween, that is, this circumstance is equivalent to that n is sufficiently small (0≦n≦1), an approximate result is determined before ruining into a proper final stage, and thus there is little place for comeback win. When a game is played by two players in which there is a little skill difference therebetween, that is, this circumstance corresponds to that n is sufficiently large, the result of the game is not clear till the last moment.

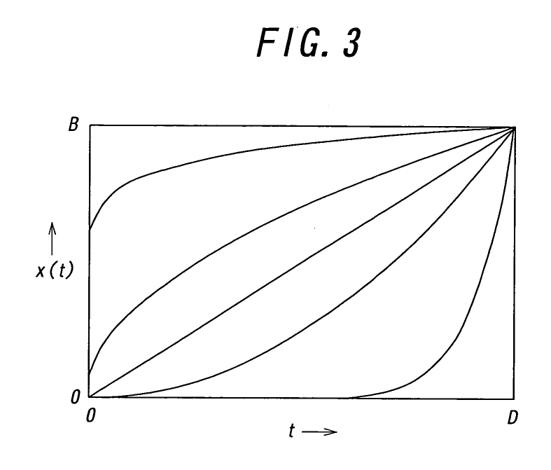

FIG. 3 illustrates this logistic model as a graph. In FIG. 3, a transition of an amount of information (vertical axis) with respect to time (horizontal axis) is represented. The amount of information x(t) is relevant to the result of the game. Since the amount of information regarding to the game result is positively correlated with options (degree of freedom or flexibility) in a given game, if the model is applied to a board game, this model can be constructed using average numbers of possible or legal moves. The equation of this logistic model includes a factor n, this factor is a parameter influencing the slope of a model line. When two players have similar playing skills of the game (i.e., when a game progresses in a pattern such as a seesaw battle), n becomes sufficiently larger than one. The lowest curved line in FIG. 3 is a model curved line in a game pattern such as the above described seesaw game. Looking from other side, the game pattern implies that information required to know the game result is uncertain until the very last minute such that the game is almost finished. In this manner curved lines in the graph are lines expressing game patterns. As shown in FIG. 3, in this logistic model, a result of moves (x(t)=b) selected by players becomes completely clear at the point of game ending (t=d). It is assumed that there exits a derived function, which is derived from x(t), in an interval [0,D] on a time scale in equation (1), a concept of derivation is introduced as follows. In addition, the present invention covers systems such as both consecutive things having continuous time and discrete things having a concept of moves or turns by each player. Here, in this context, derivative values express a speed of change of uncertainty as the game progresses (i.e., passage of time t). In other words, this speed can be qualified as increasing rate of field of view. This speed depends on kinds of games or ability of players. Equation (1) can be converted by differentiating the equation into a following first order derivative.
X′(t)=(B/Dⁿ)ntⁿ⁻¹ equation (2)

In addition, it is supposed that equation (1) can be modified into a second order derivative in an interval [0,D]. The second order derivative values express an accelerated velocity of shift of uncertainty as a game progresses. In other words, the second order derivative values can be qualified as changed portions of speed of uncertainty shift (i.e., shift of an amount of acquired information). Equation (1) is modified into a following second derivative.
x″(t)=(B/Dⁿ)n(n−1)tⁿ⁻² equation (3)

As mentioned above, a situation such that players do not know or estimate the ending of the game at the last second in the final phase of the game means that the value of second order derivative (accelerated velocity) in the proposed logistic model becomes larger. In other words, the larger this second order derivative value is in the final phase of the game, i.e., at t=d, the more exciting and the more thrilling the game is. In the scheme of this invention, such more exciting game is evaluated as a more sophisticated or refined game. Substituting t=D (i.e., final phase of the game) into equation (3), the following equation is obtained.
x″(t)=(B/Dⁿ)n(n−1)Dⁿ⁻²
x″(t)=(B/D²)n(n−1) equation (4)

Equation (4) implies that, if a skill difference between players is in a certain vicinity range (n>1), the larger a value of a refinement degree provided by this invention S=B/D² is, the more refined the game is. In like manner a square root, “B^{0.5}/D”, of that value may be used as the sophistication index.

However, it is not desirable that the sophistication degree S becomes oversized. In general, if the S is within a certain range, it can be presumed that the larger the refinement or sophistication or index S is, the more sophisticated the game is. Therefore, a degree of sophistication up to the point in which the sophistication index S gets to a certain upper limit would be increased, if the index S is beyond the certain upper limit a degree of sophistication would be reduced adversely. A value of the sophistication degree S is thought of as a reference index representing a balance between skills or abilities of players and contingencies in a given game. It has a tendency that when the game is a game depending on skills of players a value of S is decreased, and a value of S is increased when the game is a game depending on eventualities. This is raised from that average number of actual moves (i.e., a time length for coming to a conclusion) becomes large when skills of players are improved. Therefore, when a game system has a good balance between skills and eventualities, competition tends to be a seesaw battle by players having closest skills and thus it is conceived that such game system is sophisticated.

Meanwhile in self-automatic battles by computer players, which will be explained in detail later, it is envisioned that computer players have substantially the same game playing skills.

Comparison Based on the Index According to the Invention

Table 1 illustrates both data regarding information B and D about well-known games and data regarding some indexes or rates calculated by the evaluation system according to the present invention. These data are based on statistical information acquired from game records of masters for shogi and go and grand masters for chess or xiangqi.

TABLE 1

| | B | D | B/D² | B^{0.5}/D |
|---|---|---|---|---|
| Go | 250 | 250 | 0.037 | 0.061 |
| Xiangqi | 38 | 95 | 0.042 | 0.065 |
| Chess | 35 | 80 | 0.055 | 0.074 |
| Shogi | 80 | 115 | 0.060 | 0.078 |

In games for which there are masters groups and which have long histories, such as chess, shogi, xiangqi and go, it can be assumed that official game records have information that such games have been played in groups only including professional players (i.e., masters) having the closest game playing skills. Therefore, it can be evaluated that the larger value of “B/D²” the game has, the more sophisticated the game is. Based on table 1, it is found that shogi is the most sophisticated and fascinating game among them listed in table 1. Alternatively, if it is assumed that respective games have almost similar sophistication or refinement rates, it can be evaluated average performance or skill of the players group having a smaller value of the B/D². But, depending on circumstance such as a social position of players, it is possible that there are little differences between average skills of professional players groups by game. In table it is found that the index value for go is small. It can be attributed to a certain manner such that, even if the result of the game (i.e., winning or losing) becomes almost clear in the game of go, the players carry on playing of the game until the end for easy counting respective points (pieces) on the board. If such redundant parts can be eliminated from the index value, it is presumed that go has substantially the same sophistication degree of chess or shogi.

Here, number of possible moves (legal moves) in a certain game represents “number of moves” which can be played in that situation according to game rules. For example, in a game of shogi, number of possible moves is a sum of number of moves capable of moving placed pieces on the board and number of moves capable of dropping pieces in hand onto the board. Number of actual moves played in the game is number when winning or losing is determined. Since in general players give up the game for lost when they become conscious of losing, number of actual moves played in the game depends on skills of players. If a game is played between weaker players, since a balance of a game is broken or disrupted at an early stage, the number of actual moves will be decreased. On the contrary, if a game is played between stronger players, since they can become aware of losing the game and a balance is not easily disrupted, taking these things into account the number of actual play in the game will be increased. Meanwhile, number of possible moves depends partly on skills of players, but the degree of the dependence of possible moves is not high as that of that of actual moves. Therefore, the value of B^{0.5}/D of games among stronger players becomes less than that for games among weaker players according to statistics information.

For example, in fields of sport competition, length of a game has been reduced in order to sophisticate or refine a game within the limits without almost change in freedom level of a game. This example obviously shows of increasing the sophistication index. This example includes to adopt the tiebreak system in tennis and to revoke the service point system (i.e., adopting the rally point scoring system) in volleyball. In a next stage, an evolution of a game and improving skills of players (including both human beings and computer) as easy applications based on the sophistication rate or index provided by the present invention will be explained. Of course, the present invention is not limited to these applications and is widely applicable to various things.

Application to Game Evolution

In some kinds of chess games known in history, automatic battles by the computer players were carried out and game data was collected. There is a little difference between players, since the battles are self-fighting by the same two computers. The collected data is evaluated as information played by players having the same skills for every game, since the computers have been loaded the same algorithm (e.g., prediction search feature, the evaluation function learned automatically, and customized feature for extending search). Therefore, transition of the degree of refinement or sophistication in game evolution can be seen by comparing the values of sophistication rates or degrees, “B/D²” acquired by the above simulation.

TABLE 2

| | D | B | E (G) | B^{0.5}/D | B^{D} | Age |
|---|---|---|---|---|---|---|
| Chaturanga | 175.982 | 19.386 | 39.969 | 0.025 | O (225) | A.D. 4 |
| Shatranj | 222.298 | 19.180 | 50.759 | 0.020 | O (284) | A.D. 6 |
| Medieval I | 230.593 | 20.195 | 51.312 | 0.019 | O (299) | A.D. 8 |
| Medieval II | 217.458 | 20.981 | 47.475 | 0.021 | O (287) | A.D. 12 |
| Medieval III | 185.263 | 20.790 | 40.631 | 0.025 | O (241) | A.D. 15 |
| New Chess | 100.852 | 26.684 | 19.524 | 0.051 | O (143) | A.D. 16 |
| Chess | 100.060 | 26.981 | 19.263 | 0.052 | O (142) | A.D. 16 |

A value of function E(G) is an inverse number of “B^{0.5}/D” and thus the function E(G)=D/B^{0.5}. A value of B^{D} is equivalent to a size of mini-max tree which must be searched for knowing a theoretical value of the game (i.e., game result or winning or losing in an initial phase). In other words, the value of B^{D} represents “an evaluation value of complexity or difficulty of a game”. Although in general if this value becomes too large the game is complicated, redundancy is generated when the value is merely large. Therefore, the value of B^{D} can be understood as things representing “apparent complexity of the game”. Knowledge obtained from statistical information in this table is as follows:
(1) The present chess is the most refined game in this table.
(2) Games had evolved to the orientation where apparent complexity is increasing in early stages, but after that games has evolved in the direction where the degree of sophistication (it can also be called the fun of a game) is increasing.
   Application to Improvement of Player's Skills

Average number of actual moves of a game is significantly changed based on skills of a players group who are playing games but average number of possible moves is almost not influenced by such skills. Therefore, skills of game playing software or computer can be improved by automatically fighting between such computer players having similar skills (e.g., game playing computer or software such as shogi playing application software) and by adjusting such software or computer to elongate the average number of actual moves. Because the value of “B/D2” can be reduced by just increasing the value of the average number of actual moves D. In many cases, the value of “B/D2” obtained from data of professional go player groups is small, and the value obtained from data of player groups weaker than them becomes large. Conversely, it can be said that the higher the player group's skill is, the smaller the value of “B/D2” or “B0.5/D” is.

The present invention can be applicable not only to board games but also to other competition systems. In general, completion or finish time is defined as a playing time in competitive system. If the competitive system is a sport game, the completion time is defined as a time from the beginning to the end of the game. For a soccer game, if extended time is removed, the finish time is 90 minutes. The amount of information x(t) can be treated as transition of the result in a competitive system. In board games, the transition represents the grade how much the result of victory or defeat becomes clear in respect to progress of time. Therefore, if the last goal (i.e., criteria for determination of winning or losing) in an arbitrary game systems is clear, it is possible to define x(t) reasonably. For example in a soccer game, when remaining time is five minutes and there is three points difference between them, it is considered that the x(t) is determinate. Even if the remaining time is 10 minutes and there is three points difference, it is considered that the x(t) is determinate, however, it can be considered the degree of deterministic for this case is lower than that of former case.

In addition, in the case of board games, a degree of freedom can rationalistically and objectively be approximated as an average number of possible moves and thus the freedom degree can be defined. Meanwhile, in the case of other competitive systems, it is almost impossible to approximate the freedom degree to such extent for board games. However, according to the present invention, even if competitive system other than board games, the degree of freedom can be approximated or defined and thus it is possible those competitive systems are relatively compared with each other. For example, in soccer games, it is assumed that the games have the same rules and there are only differences among the areas of playing fields (each are is equivalent to the number of squares, and has a positive correlation with the number of possible moves (freedom degree or flexibility) in shogi or chess), the freedom degree or flexibility can be relatively defined and expressed numerically. The degree of freedom can relatively be defined and compared based even on with or without of the offside rule, the existence of an extra-inning game or a penalty shootout, the number of players and a difference of the number of the players who can be changed, which are equivalent to foul play or illegal play and number of pieces in shogi or chess and which have positive correlation with number of legal or possible moves (i.e., the degree of freedom). When a predetermined freedom degree criteria table is prepared to properly define points toward these sub-parameters regarding the degree of freedom, the degree of freedom can rationalistically and objectively be obtained with reference to the prepared criteria table. Therefore, a value of the degree of sophistication (B^{0.5}/D) can be calculated from the obtained degree of freedom, and competitive systems can relatively be compared among them. The competitive systems are naturally selected and evolve into systems to have adequate values of the sophistication degree. In other words, the degree of sophistication is increased to some certain extent, but if the degree of sophistication is increased too much (i.e., D is sufficiently small to B), the competitive system become have lots of elements which are influenced by coincidence and influence of player's skills is decreased in that competitive system. In this context, the competitive system will evolve to decrease the value of the sophistication degree. Namely, optimization of a balance between skills and coincidence are performed in an evolution process.

The features and effects of this invention are summarized as follows:
(i) A necessary condition in order to increase the degree of refinement of a game is that the quantity of the degree of freedom or flexibility given to players, i.e., the indefinite nature with which a game provides players, is a suitable scale and that it is preferable that the end (i.e., victory and defeat) of a game is not known to the last when the players having similar skills are battled against each other. Entertaining or fascinating aspect of competitive systems will be lost if a difference is generated to the situation in which a losing side cannot turn back.
(ii) The logistic model is provided such that the vertical axis denotes an amount of information x(t), which is needed for deterministically knowing the conclusion (i.e., winning or losing) of a game and the horizontal axis denotes time t. Additionally, in this logistic model, a reference index i.e., the degree of sophistication regarding to a game system is derived by introducing second order differentiation.
(iii) Several game records, in which the records were traditional games and there were professional game player groups or leagues for a certain period, were compared using the reference indexes provided by the present invention. If it was assumed that each player group have the same average capabilities, shogi was the most sophisticated game. If it was assumed that the sophistication degree was identical by each game, it is found that the average skills of professional player groups in go and xiangqi are slightly higher than that of professional player groups in chess or shogi. This means that go and xianqi are games more depending player's skills than that of chess and shogi.
(iv) The method of the application to chess species evolution (refinement) and the application to the improvement in skills of players (e.g., software and computer) was shown. If a player group's average capability is fixed, the degree of refinement of a game can be estimated. On the contrary, if it is assumed the degree of refinement of a game is the same (this is equivalent to that there are the same kind game and the rule of some of games are slightly changed), the average strength of a player or a player group can be presumed from average number of actual moves. When it is desired that handicap games such as starting with lesser pieces or starting with previously dropped some go pieces are played, proper conditions for that handicap battle can be estimated based on the number of actual moves.
(v) The present invention can be applicable to various things other than the above described applications. If a thing can be modeled as a game (i.e., a competitive system or an entertainment system) using both a degree of freedom and time and there is a proper validity to the thing to being sophisticated, natural selection is performed so that the indexes provided by the present invention become larger and thus the thing evolutes. For example, if there is a game, which has lost excitement or fascinating at the present day, among traditional games, it can be coped with by changing rules so that the indexes may appropriately be exactly adjusted for that game.

In consideration of the various embodiments of the systems or methods according to the invention, the principle of the invention can widely be applied.

Further, it should be understood that the detailed description and specific examples, while indicating preferred embodiments of the invention, are given by way of illustration only, not to be used to interpret the scope of the invention. Various changes and modifications within the spirit and scope of the invention will become apparent to those skilled in the art from this detailed description.

1. A measurement system for measuring a refinement rate of a competitive system, said system comprising:
input means for inputting competition data of the competitive system, said competition data, obtained by users between a beginning and end of the competition, including transition in information amount necessary to know a conclusion of the competition;
first calculating means for obtaining an average completion time length D and an average degree of freedom B of the user and from said inputted competition data; and
second calculating means for calculating a refinement rate S based on said obtained B and D.

2. The measurement system according to claim 1, wherein said second calculation means calculates the refinement rate S using following equations:
S=B/D² or S=B^{0.5}/D.

3. The measurement system according to claim 1, wherein said, input means inputs the competition data by each user group,
and wherein said second calculating means calculates said refinement rate S as a skill level J by each user group.

4. The measurement system according to claim 1, wherein said competitive system is a board game; said average degree of freedom B is an average number of possible moves between the beginning and the end of the game; and said average completion time length D is an average number of actual moves played in the game.

5. A measurement method for measuring a refinement rate of a competitive system, the method comprising the steps of:
inputting competition data of the competitive system, said competition data, obtained by users between a beginning and end of the competition, including transition in information amount necessary to know a conclusion of the competition;
obtaining an average completion time length D and an average degree of freedom B of the user and from said inputted competition data using calculating means; and
calculating a refinement rate S based on said obtained B and D using calculating means.

6. The measurement method according to claim 5, wherein said calculating step calculates the refinement rate S using following equations:
S=B/D² or S=B^{0.5}/D.

7. The measurement method according to claim 5, wherein said inputting step inputs the competition data by each user group,
and wherein said calculating step calculates said refinement rate S as a skill level J by each user group.

8. The measurement method according to claim 5, wherein said competitive system is a board game; said average degree of freedom B is an average number of possible moves between the beginning and the end of the game; and said average completion time length D is an average number of actual moves played in the game.

9. A measurement program for executing a measurement method for measuring a refinement rate of a competitive system, the program comprising the steps of:
inputting competition data of the competitive system, said competition data, obtained by users between a beginning and end of the competition, including transition in information amount necessary to know a conclusion of the competition;
obtaining an average completion time length D and an average degree of freedom B of the user and from said inputted competition data; and
calculating a refinement rate S based on said obtained B and D.

10. The measurement program according to claim 9, wherein said calculating step calculates the refinement rate S using following equations:
S=B/D² or S=B^{0.5}/D.

11. The measurement program according to claim 9, wherein said inputting step inputs the competition data by each user group,
and wherein said calculating step calculates said refinement rate S as a skill level J by each user group.

12. The measurement program according to claim 9, wherein said competitive system is a board game; said average degree of freedom B is an average number of possible moves between the beginning and the end of the game; and said average completion time length D is an average number of actual moves played in the game.

13. The measurement system according to claim 2, wherein said competitive system is a board game; said average degree of freedom B is an average number of possible moves between the beginning and the end of the game; and said average completion time length D is an average number of actual moves played in the game.

14. The measurement system according to claim 3, wherein said competitive system is a board game; said average degree of freedom B is an average number of possible moves between the beginning and the end of the game; and said average completion time length D is an average number of actual moves played in the game.

15. The measurement method according to claim 6, wherein said competitive system is a board game; said average degree of freedom B is an average number of possible moves between the beginning and the end of the game; and said average completion time length D is an average number of actual moves played in the game.

16. The measurement method according to claim 7, wherein said competitive system is a board game; said average degree of freedom B is an average number of possible moves between the beginning and the end of the game; and said average completion time length D is an average number of actual moves played in the game.

17. The measurement program according to claim 10, wherein said competitive system is a board game; said average degree of freedom B is an average number of possible moves between the beginning and the end of the game; and said average completion time length D is an average number of actual moves played in the game.

18. The measurement program according to claim 11, wherein said competitive system is a board game; said average degree of freedom B is an average number of possible moves between the beginning and the end of the game; and said average completion time length D is an average number of actual moves played in the game.
